(19) United States
(12) Patent Application Publication
(10) Pub. No.: US 2016/0021425 A1
(43) Pub. Date: Jan. 21, 2016

(54) SYSTEM AND METHOD FOR PREDICTING AUDIENCE RESPONSES TO CONTENT FROM ELECTRO-DERMAL ACTIVITY SIGNALS

(71) Applicant: THOMSON LICENCING, Issy les Moulineaux (FR)

(72) Inventors: Brian ERIKSSON, San Jose, CA (US); Fernando Jorge SILVEIRA-FILHO, San Francisco, CA (US); Anmol SHETH, San Francisco, CA (US)

(73) Assignee: Thomson Licensing

(21) Appl. No.: 14/773,409

(22) PCT Filed: Mar. 10, 2014
(86) PCT No.: PCT/US2014/022275

§ 371 Date: Sep. 8, 2015

Related U.S. Application Data
(60) Provisional application No. 61/839,669, filed on Jun. 26, 2013.

(51) Int. Cl. H04N 21/466; A61B 5/16; H04N 21/25; H04N 21/442; H04N 21/258; A61B 5/053; A61B 5/00

(52) U.S. Cl. CPC H04N 21/4665; A61B 5/0533; A61B 5/165; A61B 5/7264; H04N 21/44218; H04N 21/4667; H04N 21/25883; H04N 21/252

(57) ABSTRACT

A method for determining user responses to content commences by collecting Electro-Dermal Activity (EDA) signals from a user via a collection system as the user consumes (e.g., views) the content. From the collected EDA signals, the amplitudes of the users' responses are extracted at particular times. The extracted amplitudes undergo processing with demographic information for the user and parameters of the collection system obtained during training to predict feedback of the user to the content.

### CROSS-REFERENCE TO RELATED APPLICATIONS

This application claims priority under 35 U.S.C. 119(e) to U.S. Provisional Patent Application Ser. No. 61/839,669 filed Jun. 26, 2013, the teachings of which are incorporated herein.

### TECHNICAL FIELD

This invention relates to a technique for assessing users' responses to content in accordance with electro-dermal activity signals.

### BACKGROUND ART

Assessing the reaction of viewers to content they consume has importance for a wide variety of applications. Examples of such applications range from movie recommendation systems, which utilize user reaction to obtain user's preferences, to market research, where content creators conduct surveys and focus groups with test audiences to predict the success of movie productions or ad campaigns. While these applications traditionally obtain explicit user feedback via ratings and survey forms, numerous factors constrain these traditional approaches for gathering user feedback. For example, existing movie recommendation systems request viewers provide only a single rating for the entire movie. Survey forms have space limitations and rely on viewer memory, which fades over time. Participation costs and time limitations constrain the use of focus groups. Thus, traditional approaches for gathering user feedback do not afford detailed (e.g., “fine grain”) user response to content.

The advent of wearable biometric sensors now enables capturing user's responses to content with much finer granularity than past techniques. Consumer electronic equipment like watches and fitness devices now include embedded biometric sensors for heart rate and Electro-Dermal Activity (EDA) for continuously monitoring the physiological responses of the user. Such consumer electronic equipment record EDA as the conductance between a pair of electrodes placed over a user's skin near concentrations of sweat glands, hereinafter referred to as Skin Conductance Response or SCR. An individual's EDA has a well-known correlation to brain activation from emotional reactions to stimulus, which causes sudomotor neuron bursts and results in the expulsion of sweat from eccurine glands, causing conductance variations across the individual's skin.

Scientists have studied the psychological correlation between an individual's emotional reactions and resultant changes in EDA since the early 20th century. Signals generated from EDA provide a rich source of implicit feedback useful for inferring individuals' reactions to content at various granularities. Unfortunately, no straightforward method presently exists for direct inference of user opinion of content using EDA signals. Current approaches suffer from several important challenges. Signals obtained from EDA carry noise and stimuli not part of the content, e.g., distractions in the environment will adversely affect such signals. Additionally, the responses contained within the signals vary considerably based on the type of stimuli. Further, such responses depend on the individual's physiological and psychological state. Various other factors also complicate EDA signal interpretation, such as potentially overlapping events, attenuation of event activity amplitude for repeated stimulus, varying sweat burst responses, and underlying these factors, slowly varying, skin conductance levels.

Thus, a need exists for a technique for assessing fine-grain user responses from EDA signals.

### BRIEF SUMMARY OF THE INVENTION

Briefly, in accordance with the present principles, a method for determining user responses to content commences by collecting Electro-Dermal Activity (EDA) signals from a user via a collection system as the user consumes (e.g., views) the content. From the collected EDA signals, the amplitudes of the users' responses are extracted at particular times. The extracted amplitudes undergo processing with demographic information for the user and parameters of the collection system obtained during training to predict feedback of the user to the content.

### BRIEF DESCRIPTION OF THE DRAWINGS

FIG. 1 depicts a block schematic diagram of a system for collecting EDA signals from a plurality of users during system training;

FIG. 2 depicts the system of FIG. 1 during acquisition of EDA signals from a single user for estimating feedback of that user to the content;

FIG. 3 depicts in flowchart form the steps of a method for processing EDA signals to predict feedback of the user to the content;

FIG. 4 depicts a graph illustrating exemplary EDA signals of a single user over time;

FIG. 5 depicts an exemplary sensor for measuring EDA signals;

FIG. 6 depicts a graph illustrating EDA signals from multiple users over time to different content as part of the training of the system of FIG. 1;

FIG. 7 depicts a graph of Skin Conductance Response (SCR) over time for different SCR shapes; and

FIGS. 8A and 8B depict EDA signal responses from users as point intensities for two scenes from two separate movies.

### DETAILED DESCRIPTION

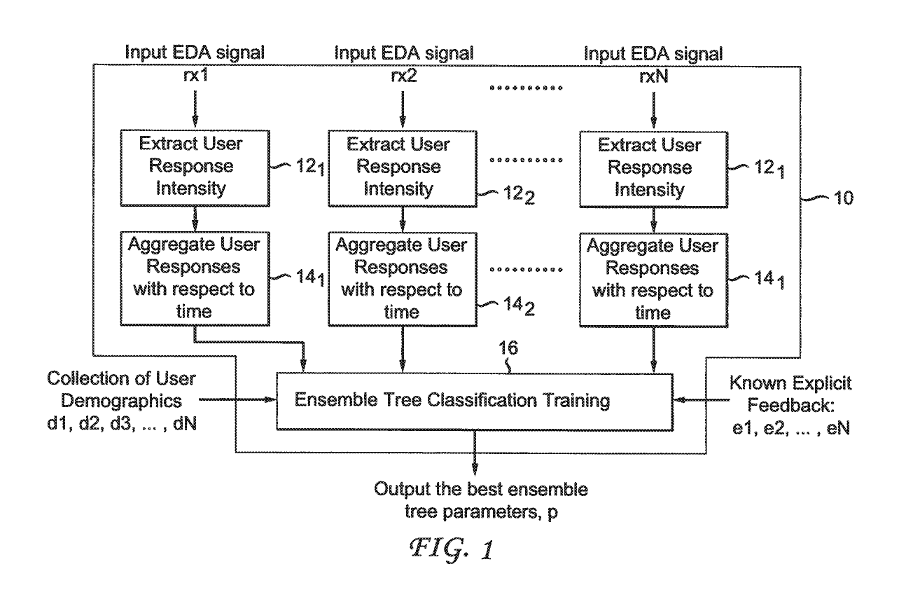

FIG. 1 depicts a system 10 in accordance with a preferred embodiment of the present principles for estimating user feedback to content by collecting and processing Electro-Dermal Activity (EDA) signals from the user during content consumption. In practice, the content takes the form of an audio-visual presentation, such as a movie or television program containing both video and audio, which the user consumes by viewing. However, the user feedback estimation technique of the present principles has applicability to other forms and types of content not including video and/or audio.

The system 10 of FIG. 1 typically takes the form of a computer, e.g., a personal computer, comprising a processor, memory, a display, and one or more data input/output devices (e.g., a keyboard and mouse and/or touch screen), as well as a network interface card, all not shown, but well-known in the art. To estimate user feedback to content, the system 10 first undergoes training by first collecting EDA signals from a plurality of users, along with demographics of those users and explicit user feedback to estimate (e.g., learn) system parameters later used in connection with the analysis of EDA signals of for an individual user. As described hereinafter, the system 10, once trained, can map EDA signals to expected explicit user feedback to extrapolate explicit feedback of users for whom the system 10 has only obtained biometric data (e.g., EDA signals).

As discussed in detail hereinafter, the system 10, in accordance with another aspect of the present principles, can process multiple streams of EDA signals from individuals as they consume content. The system 10 can capture these streams in parallel for real-time analysis for a whole audience who consume the content simultaneously, or during multiple sessions with separate groups of individuals for offline analysis. Stream synchronization occurs using external methods (e.g., marking the EDA signals) with reference to a known event, such as the beginning of the movie.

Referring to FIG. 1, training of the system 10 occurs by first receiving raw EDA signals (rx1, rx2, . . . rxN) from N users u₁-u_{N}, respectively, where N constitutes an integer greater than 1. The system 10 also receives demographic information (d1, d2 . . . dN) from the N users, as well as responses (e1, e2, . . . eN) from the N users to explicit feedback questions. The system 10 then pre-processes the raw EDA signals (rx1, rx2, . . . rxN) from the N users at a corresponding one of blocks 12₁, 12₂ . . . 12_{N}, respectively, using one or more methods (e.g., deconvolution, change-point detection, or adaptive decomposition) to extract the amplitudes of each user's responses at particular time points. In practice, the blocks 12₁, 12₂ . . . 12_{N} correspond to separate processing cycles of a single processor with each cycle corresponding pre-processing of an individual signal. However, the blocks 12₁, 12₂ . . . 12_{N} could comprise individual hardware elements (or hardware elements that execute software) for performing signal amplitude extraction. The signal amplitudes extracted by each of the blocks 12₁, 12₂ . . . 12_{N} undergoes aggregation for relevant time-segments of the stimulus (typically through simple addition of amplitudes) at a corresponding one of blocks 14₁, 14₂ . . . 14_{N}, respectively. Like the blocks 12₁, 12₂ . . . 12_{N}, the blocks 14₁, 14₂ . . . 14_{N} correspond to separate processing cycles of a single processor, but could represent separate hardware elements for performing amplitude aggregation.

At this point, the system 10 now has for each user: (1) demographic information; (2) extracted and aggregated EDA responses collected with respect to the stimulus (e.g., the consumed content); and (3) known explicit user feedback. Using the aggregated EDA signal amplitudes from the blocks 14₁-14_{N}, the system 10 establishes a set of parameters p of for a set of ensemble classification trees at block 16 to predict content ratings from EDA signals collected from users. The block 16 typically corresponds one or processing cycles of the processor but could comprise a separate hardware element.

Each classification tree constitutes a model that predicts a value of a target variable based on the value of various input variables. Each tree has one or more interior nodes, each node corresponding to an input variable. Each node has one or more edges (branches) that represent paths taken in the tree based on the value of the input variable at that node. Each path terminates at a “leaf” that represents the value of a target variable resulting from the value of the input variable. In accordance with an aspect of the present disclosure, the system 10 thus trains itself, thereby creating the ensemble classification parameters (p) by learning from: (1) demographics information; (2) extracted and aggregated EDA responses collected with respect to the stimulus; and (3) known explicit feedback of that user. Using trained parameters (p), the system 10 can determine subsets of variables (i.e., aggregated EDA user responses and demographics) relevant for discriminating among explicit users feedback.

In accordance with another aspect of the present principles, the system 10 of FIG. 1 advantageously addresses the above-described problems involved in interpreting EDA signals. As described hereinafter, the system 10 of FIG. 1 can infer user opinion of consumed content using physiological signals by a “Greedy” algorithm matching pursuit to extract the relevant impulse information and by adapting to changing physiological environments using a construction of possible user EDA responses. To this end, the system 10 requires only the raw EDA signal identifying the time, location, and intensity of user responses.

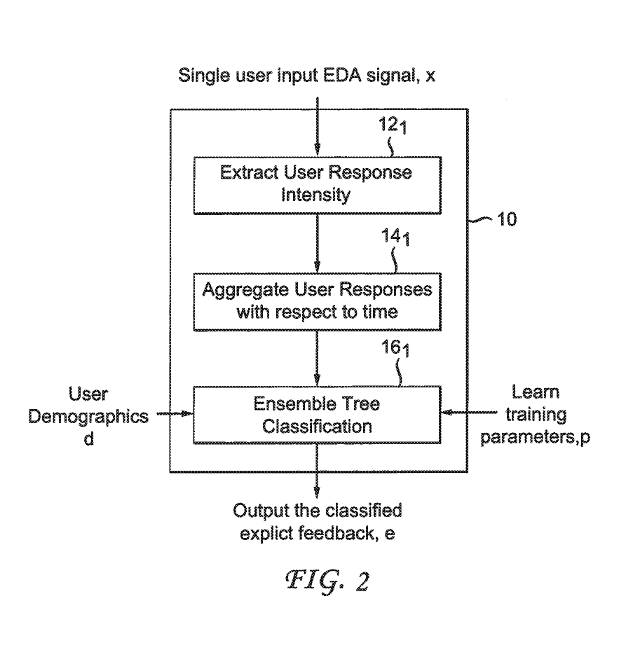

In accordance with another aspect of the present principles, the system 10 can make use of a user's (1) EDA signals, and (2) demographics information, along with (3) learned system parameters to infer unknown explicit feedback of a user for whom the system 10 has only collected EDA signals. To better understand the manner in which the system 10 make such inference, refer to FIG. 2 which depicts a portion of the system 10 including a single block 12₁ for extracting the amplitude of the EDA signal for the user u₁ at particular time points. Signal amplitude extraction in FIG. 2 occurs in the same manner in which EDA signal amplitude extraction occurs for multiple users in FIG. 1. The block 14₁ aggregates the extracted EDA signal amplitude for the single user u₁ for relevant time-segments of the stimulus (typically through simple addition of amplitudes), similar to the manner in which EDA signal amplitude aggregation occurs in FIG. 2 for multiple users. Lastly, the block 16₁ of FIG. 2 performs of ensemble tree classification to predict the explicit feedback of the user u₁ based on the aggregated EDA signal amplitude, the demographics d₁ for the user u₁ and the learned training parameters p obtained in connection with training of the system as described with respect to FIG. 1.

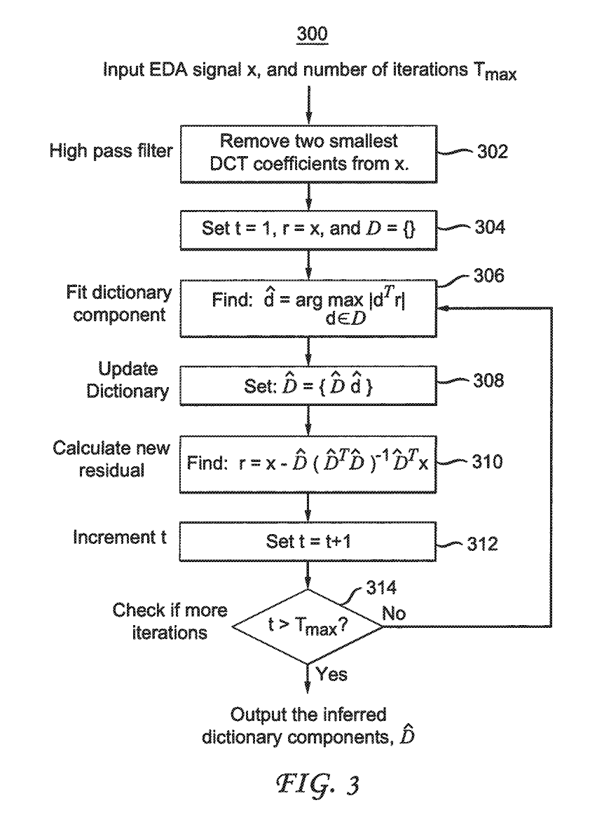

FIG. 3 depicts in flow chart form the steps of on an exemplary process 300 in accordance with a preferred embodiment of the present principles for execution by the system 10 of FIG. 1 to predict the explicit feedback for the user u₁. As discussed above in connection with FIG. 2, once trained, the system 10 will collect the EDA signals from the single user u₁ during content consumption or other stimulus for observation and evaluation. The system 10 decomposes the EDA signal to obtain both the time of this user's reaction to the stimulus, and the magnitude of these reactions. The system 10 receives as an input the observed galvanic skin response (GSR) in the form of the raw EDA signal rx, and the maximum number of user reaction components to extract, Tₘₐₓ.

The method of FIG. 3 commences by considering the slowly varying DC component of each viewer's EDA signals. Often called the “tonic” signal, this signal component arises from the physiological response to sweat saturation-levels of the user's skin and has little correlation with the underlying fine-scale user's reactions desired for detection. In accordance with the present principles, this signal component undergoes high pass filtering during step 302 to subtract the signal contribution related to the two coarsest scale coefficients of a discrete-cosine transform (DCT) performed on the signal rx. The remaining high-pass filtered EDA signal bears the designation x (as opposed to initially collected raw EDA signal rx). Next, the signal undergoes decomposition using a large dictionary of feasible user response shapes. As described hereinafter, the consideration of many different signal types, with varying durations and decay characteristics, allows a better fit to the observed skin conductance.

Equation 1 can parameterize the specific dictionary basis functions as follows:

$${d}_{{λ}_{1} , {λ}_{2} , {t}_{0}} ( t ) = \{ \begin{matrix}{λ}_{1}^{- {λ}_{2} ( t - {t}_{0} )} & t \geq {t}_{0} \\ 0 & t < {t}_{0}\end{matrix} \qquad ( Equation 1 )$$

such that λ₁ relates to the geometric decay of the impulse, λ₂ constitutes the log-linear decay slope, and t₀ corresponds to the response start. From empirical examination of the EDA signals, the system 10 constructs the signal dictionary, D occurs using all signals for the parameter space,

λ₁ε{1.1,1.25,1.5,1.75,2,2.5,e},

λ₂ε{0.3,0.5, . . . ,3.7,3.9}. (Equation 2)

To represent each EDA signal from this large collection of dictionary signals requires solving a standard linear inverse problem. Unfortunately, using ordinary least squares approaches will consume very large amounts of memory for large dictionaries, and will also destroy the inherent desired sparsity of the SCR event process. Using an orthogonal matching pursuit technique (a greedy algorithm) to resolve the set of dictionary components that best describe the observed EDA trace will avoid such limitations.

This matching procedure begins with the raw EDA signal, rx, a signal component dictionary D (constructed using the equation above), and an empty constructed dictionary, \hat{D}={ }. During step 304, the system 10 sets the high-pass filtered EDA signal becomes such that r=x. During step 306, the system 10 determines the single dictionary component that best fits the observed EDA signal using the relationship set forth in Equation (3):

$$\hat{d} = arg {max}_{d ∈ D} \vert {d}^{T} r \vert \qquad ( Equation 3 )$$

During step 308, the system 10 updates the dictionary by adding this dictionary component to the inferred dictionary

(\hat{D}={\hat{D}\hat{d}}) (Equation 4).

During step 310, the system 10 removes contributions of this dictionary component from the observed EDA signal, creating a new residual signal in accordance with Equation 5:

r=x−\hat{D}(\hat{D}^{T}\hat{D})⁻¹\hat{D}^{T}x. (Equation 5)

This process repeats for a specified number of iterations by first incrementing a time value t by unity during step 312 and then determining during step 314 whether the value of t exceeds a maximum time value Tₘₐₓ. If so, the process ends. If not, the process 300 branches to step 306. Performing the desired number of iterations thus yields a collection of dictionary components that fits to the observed signal. In summary, for each EDA signal of a given user, the adaptive decomposition approach of the process 300 executed by the system 10 yields a collection of user reaction dictionary components, represented by a set of time offsets (the time-start of each occurrence of a dictionary component) and the coefficient amplitudes of the user response events, respectively.

The system 10, as thus described, addresses the challenge of obtaining fine-grain user responses by using electro-dermal activity (EDA) signals of users consuming content and accurately mapping such signals to self-reported explicit feedback provided by such users. This approach not only improves existing approaches to calibrate audience feedback, but also enables a range of new applications such as indexing and searching individual content, and providing content recommendation systems that can propose content that best matches the physiological state of the user. To this end, the system 10 advantageously decomposes raw EDA signals (rx) into responses that accurately pinpoint the times and intensities of viewer responses to the stimuli in the content. Further, the system 10 provides a machine-learning framework that uses the EDA responses to accurately predict the explicit feedback provided by a user.

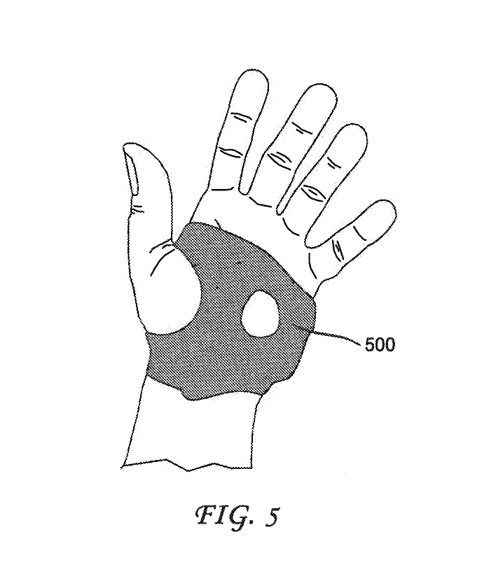

In accordance with another aspect of the present principles, the system 10 can advantageously characterize the changes in user electro-dermal activity (EDA) as such users respond to stimuli during content consumption. In this regard, the system 10 can accurately map implicit EDA feedback to the explicit feedback provided by the viewers in the form of ratings and survey forms. To that end, the system 10 can make use of one or more EDA sensors, such as the EDA sensor 500 of FIG. 5 described hereinafter, which a user wears while consuming content (e.g., watching a movie or television program).

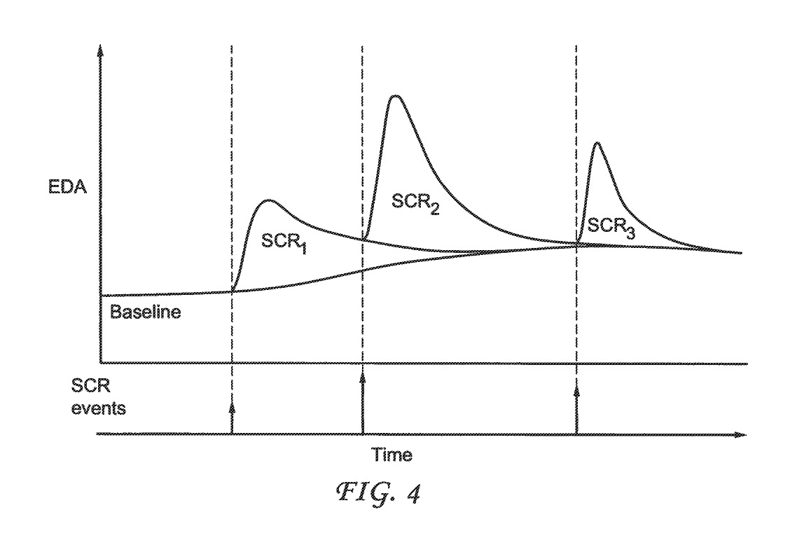

The system 10 of FIGS. 1 and 2 typically records EDA as the conductance between a pair of electrodes placed over an individual's skin, near concentrations of sweat glands. An EDA signal characteristically exhibits a slow frequency baseline component plus short-lived spike-like events, denoted Skin Conductance Responses (SCRs), which often overlap with each other, as illustrated in FIG. 4. An individual's EDA signal has a well-known connection to the brain activation resulting from emotional reactions to stimulus, which causes sudomotor neuron bursts and results in sweat to expelled from eccurine glands, finally causing conductance variations on the individual's skin. Understanding of these phenomena has increased from an examination of brain function via functional Magnetic Resonance Imaging (fMRI) and skin conduction via EDA simultaneously, showing the activations in specific regions of the brain that result in variations in the EDA. In addition, micro-video recordings of sweat glands clearly demonstrate that neuron firings result in variations in skin conductance. Scientists have conducted extensive work in evaluating the connection between SCRs and activities such as video game playing, performing arts viewing, everyday interactions, detecting stress, evaluating cognitive load, and determining perception changes due to mental illness.

In accordance with another aspect of the present principles, the system 10 has the capability of analyzing user EDA signal responses to stimuli (e.g., content viewing). FIG. 4 shows an example of an EDA signal with decomposed the Skin Conductance Response (SCR) events, thus illustrating the challenges involved in characterizing SCR events from a raw EDA signal. Specifically, extraction of true user neuron burst events from EDA signals often proves difficult because of potentially overlapping events, attenuation of event amplitude for repeated stimulus, varying burst impulse functions, and underlying all these, slowly varying skin conductance levels. Various proposed signal decomposition approaches to combat such difficulties include highly parametric sigmoid-exponential model, bi-exponential impulse responses, nonnegative deconvolution, and Variational Bayesian decomposition techniques. These techniques incur limitations either as a result of computational complexity, inability to discover overlapping events, or a one-size-fits-all approach not sufficiently robust to accommodate varying event durations. In accordance with an aspect of the present principles, the system 10 employs a matching pursuit-based methodology to extract relevant impulse information with low computational complexity and high adaptivity to changing physiological environments. Inputs comprise the raw EDA signal and the system 10 identifies both the time and intensity of SCR events.

In accordance with another aspect of the present principles, the system 10 can advantageously predict explicit feedback from EDA signals and address the problem of assessing user reactions to stimulus (e.g., view content) using EDA signals. In contrast to other approaches that focus on isolated experiments on individual users, the system 10 advantageously provides concurrent, audience-level evaluation of SCR events previously decomposed by the signal processing method described above.

In accordance with another aspect of the present principles, the system 10 advantageously processes EDA signals collected from viewers consuming (e.g., viewing) different types of audio-video content. In particular, the system 10 has successfully to collected EDA signals from an audience at scale in an environment with minimal distractions from external stimuli. In this regard, the system 10 has collected data in commercial movie theaters while audience members viewed feature-length films. The controlled temperature, lighting and immersive nature of a movie theatre enabled measuring EDA signals that mainly represented user reaction to stimuli in the movie. In addition to EDA signals, the system 10 collected explicit feedback from the audience for mapping the implicit feedback in EDA responses to the explicit feedback.

As mentioned previously, FIG. 5 shows an exemplary embodiment 500 of an EDA sensor suitable for use in accordance with principles of the present disclosure. In practice, the sensor 500 comprises a commercially available EDA sensor sold by Affectiva, Waltham Mass., which users wear on their palms. Unlike medical grade EDA sensors that typically require wired connections and conductive gel to improve signal quality, the Affectiva sensor wears easily and enables setup for a large group of study participants (between 20-30 participants) within a short time span (15-20 minutes).

As discussed above, the system 10 of FIGS. 1 and 2 performs two types of data collection operations: (1) data collection for calibration of the system and (2) data collection for sensing actual user responses to content. For example, during the second data collection operation, the system 10 can collect responses from one or more users during viewing of feature-length films. In contrast, during the data collection associated with system learning (system calibration), the system 10 monitors participants in isolation as they view content for short duration, e.g., a video clip or audio clip, with controlled audio and image stimuli for validating the system's ability to detect individual user responses.

During each data collection operation described above, the system 10 obtains raw EDA signals from the users wearing sensors, such as the sensor 500 depicted in FIG. 5. The system 10 synchronizes and pre-processes all raw EDA signals as described with respect to FIG. 1. In this regard, the processor within the system 10 will synchronize the clocks associated with the sensors prior to each recording session and the clock (not shown within the processor of the system 10 will serves to designate the beginning and ending times of the each data collection session. The sensor 500 of FIG. 5 typically measures raw skin conductance levels at 32 Hz. Given the typical duration of user skin conductance responses, the system 10 down-samples these signals to 4 Hz.

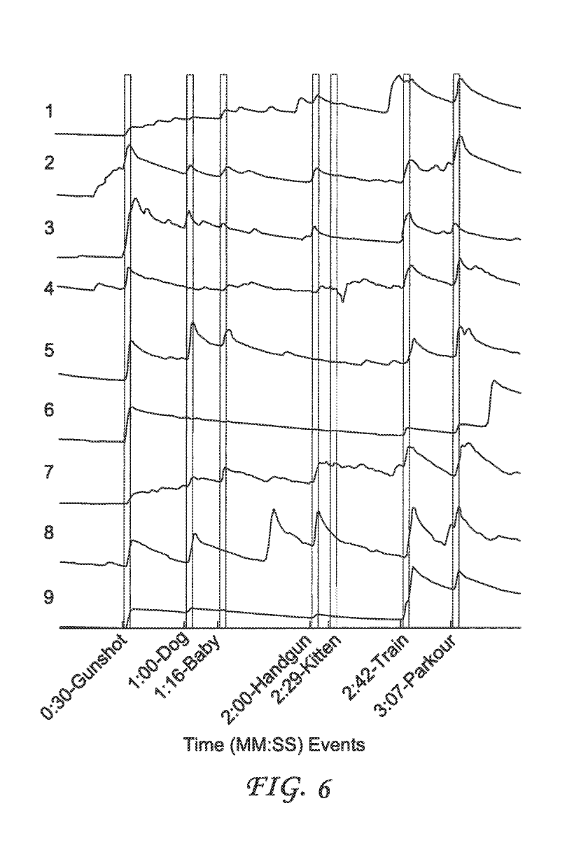

FIG. 6 graphically depicts individual EDA signals from users generated during the above-mentioned first data collection operation associated with learning by the system 10. The graph of FIG. 6 plots the EDA signals from each of nine individual users over time in response to content of varying levels of complexity. The content employed in connection with the responses depicted in FIG. 6 comprised a 220-second clip containing seven isolated stimulus events. Initially, the content provided three successive sound clips of a gunshot, a dog barking, and the a baby crying. Following the depiction of a baby crying, the content displayed the image of gun for 5 seconds, followed by the image of a kitten held appearing on the screen for the same amount of time. Finally, two short-duration (<5 seconds) video clips of near-death experiences appeared in succession, the first being a woman almost hit by an on-coming train, and second, an attempt at “parkour” ending with the individual falling face-first onto concrete. Before each stimulus, silent intervals appeared with no presented content.

The EDA signals depicted in FIG. 6 correspond to an exemplary calibration operation which collected EDA signals from nine individuals (6 male, 3 female, aged between 20 and 50 years old) who watched the content described above in isolation in a controlled laboratory environment. The EDA signals of the participants generated in response to the above-described content appear in FIG. 6 with the various stimulus events in the content marked in vertical lines.

An example of the results obtained during an exemplary second data collection operation appear in Table 1 below. The data collection operation represented in Table 1 resulted from three separate audiences viewing three feature-length films labeled A through C herein. The movies A-C had different genres (e.g., drama, thriller, foreign) to avoid limiting the scope of data collection to genre-specific phenomena. Participants in the data collection operation comprised individuals solicited from the movies' regular audiences who signed a consent form before participating.

TABLE 1

| Movie | Genres | Runtime (min) | Release | Viewers | Location |
|---|---|---|---|---|---|
| A | Action, | 130 | 2012 | 9 | Theater |
| | Crime, | | | | |
| | Thriller | | | | |
| B | Drama | 139 | 2012 | 10 | Theater |
| C | Drama, | 126 | 2011 | 15 | Film |
| | Foreign | | | | Festival |

Table 2 shows the demographics of the participants of each screening.

TABLE 2

| | Gender | | Age | | | Rating | | | | |
|---|---|---|---|---|---|---|---|---|---|---|
| Movie | Male | Female | 20-29 | 30-49 | >49 | 1 | 2 | 3 | 4 | 5 |
| A | 5 | 4 | 4 | 3 | 2 | 0 | 0 | 6 | 3 | 0 |
| B | 4 | 6 | 4 | 3 | 3 | 0 | 0 | 2 | 3 | 5 |
| C | 7 | 8 | 7 | 5 | 3 | 0 | 0 | 3 | 5 | 7 |

In addition to the audience-wide EDA signals collected for implicit audience feedback, participants were also asked to provide explicit feedback at the end of each movie screening. The explicit feedback provided input data that enabled mapping the implicit feedback in the EDA signals to the explicit feedback. The collection of explicit feedback entailed distributing survey forms to the participants that asked for the participants to provide: (1) their gender and age, and (2) an overall rating for the movie based on a 5-point scale. The survey left interpretation of what this rating implied (e.g., enjoyment, engagement, etc.) up to the user's discretion.

Advantageously, the system 10 of the present principles makes use of an adaptive decomposition methodology which processes raw EDA signals to extract precise SCR events showing exactly when and how much the viewer responds to a stimulus. As depicted in FIG. 4, identifying the relevant SCR events from raw EDA signals proves challenging because (1) SCRs may overlap, (2) they have varying duration, and (3) such SCRs may lack any correlation with the underlying stimulus (e.g., the viewer has become distracted from the stimulus). Additionally, comparing EDA signals from multiple people can also prove problematic due to varying levels of signal normalization, non-standard reaction impulse response magnitude, and differing susceptibility to react due to the deviations in the user's psychology and physiology.

In accordance with the present principles, the system 10 addresses the aforementioned problems by performing signal decomposition that automatically adapts to the variations in the user's physiology. The signal decomposition performed by the system 10 takes account of the varying DC component of each user's signal. Often called the “tonic” signal, this component corresponds to the user's physiological response to sweat saturation-levels of the user's skin and has little correlation with the underlying fine-scale user reactions of interest. As discussed previously in connection with the flow chart of FIG. 3, the system 10 removes this component by subtracting the signal contribution related to the two coarsest-scale coefficients of a discrete-cosine transform (DCT), thus yielding a high-pass, processed EDA signal that bears the designation x. Further, as discussed previously, the system 10 advantageously decomposes the resultant EDA signal using a large dictionary of feasible SCR shapes. The consideration of many different signal types, with varying durations and decay characteristics, allows a better fit to the observed skin conductance.

The specific dictionary basis functions can be parameterized by:

$${d}_{{λ}_{1} , {λ}_{2} , {t}_{0}} ( t ) = \{ \begin{matrix}{λ}_{1}^{- {λ}_{2} ( t - {t}_{0} )} & t \geq {t}_{0} \\ 0 & t < {t}_{0}\end{matrix} . \qquad ( 1 )$$

such that λ₁ relates to the geometric decay of the impulse, λ₂ is the log-linear decay slope, and t₀ is the response start. From empirical examination of EDA signals, the system 10 constructs the signal dictionary, D, using all signals d_{λ}_{₁}_{,λ}_{₂}_{,t}_{₀}(t) for:

λ₁ε{1.1,1.25,1.5,1.75,2,2.5,e}, (2)

λ₂ε{0.3,0.5, . . . ,3.7,3.9}. (3)

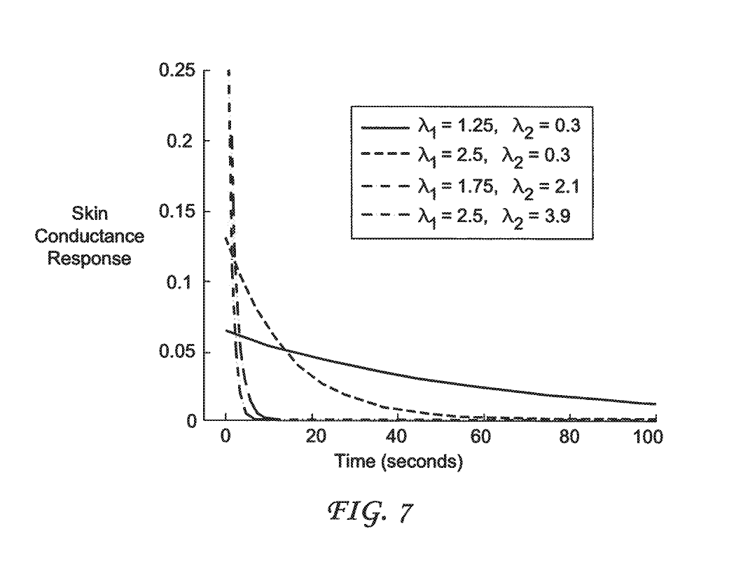

FIG. 7 depicts a plot of skin conductance response versus time for different values of this constructed dictionary for t₀=0. To represent each EDA signal from a large collection of dictionary values requires solving a standard linear inverse problem. Unfortunately, ordinary least squares approaches will require large amounts of memory for large dictionaries and destroy the inherent desired sparsity of the SCR event process. The system 10 avoids these limitations by using an orthogonal matching pursuit technique to greedily resolve the set of dictionary components that best describe the observed EDA signal.

Specifically, this matching pursuit procedure begins with the high-pass filtered EDA signal x, a signal component dictionary D constructed using Equation 1, and an empty constructed dictionary \hat{D}={ }. First, the system 10 determines the single dictionary component (\hat{d}εD) that best fits the observed EDA signal:

$$\hat{d} = arg {max}_{d ∈ D} \vert {d}^{T} x \vert . \qquad ( 4 )$$

The system 10 adds this dictionary component to the constructed dictionary \hat{D}={\hat{D}, \hat{d}}, and then removes the contributions of this dictionary component from the observed EDA signal, creating a new residual signal:

r=−\hat{D}(\hat{D}^{T}\hat{D})⁻¹\hat{D}^{T}x. (5)

The system 10 repeats this process using the residual signal (i.e., setting x=r) for a specified number of iterations.

After completing the desired number of iterations, the system 10 obtains a collection of dictionary components that fits to the observed signal. Using standard least squares, the system 10 calculates the best coefficient vector β such that the observed EDA signal is represented by a combination of elements from the constructed dictionary, x≈\hat{D}β, where the amplitude of the non-zero elements of β correspond to the intensity of user's reactions.

In summary, for each EDA signal, the adaptive decomposition approach performed by the system 10 returns, {tᵢ, sᵢ}, the set of time offsets (i.e., the time-start of each SCR event) and the coefficient amplitude of SCR events (i.e., the intensity of the SCR event), respectively.

As discussed previously, the system 10 advantageously accomplishes machine learning to predict explicit feedback of users to content (e.g., of movie ratings) from the decomposed SCR events provided by an EDA signal decomposition in accordance with the present principles. The ground-truth data of ratings for the movie comes from the user surveys taken immediately following content consumption (e.g., film viewing).

The prediction accuracy of the system 10 was compared to the accuracy achieved by using the demographic information provided by the users, e.g., age and gender information provided a set of the study participants. Table 2 summarizes the results of such a study for thirty-four study participants along with their demographic information for three films. While the comparison against demographic information may seem naive, movie studios produce feature-length films refined to target specific demographic groups. Therefore, an expectation exists for a large correlation between demographics and the resulting user responses to the films.

In the course of decomposing the SCR data of users, the system 10 obtains time-stamp and coefficient values of the SCR events for each user of length T (where T>>N). From this information, the system 10 constructs an [N×T]-implicit user response matrix S, such that the matrix element, S_{i,t}_{_{i,j}}=s_{i,j}, wherein s_{i,j} represents the user uᵢ's estimated response based on the EDA signal decomposition at time j.

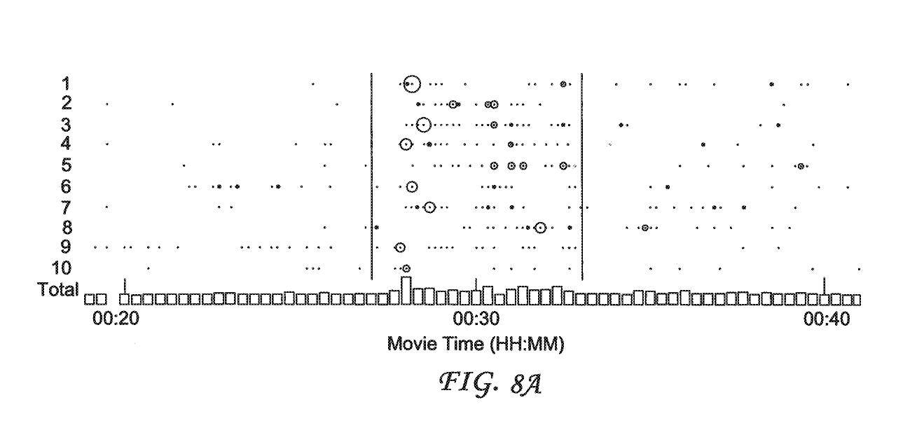

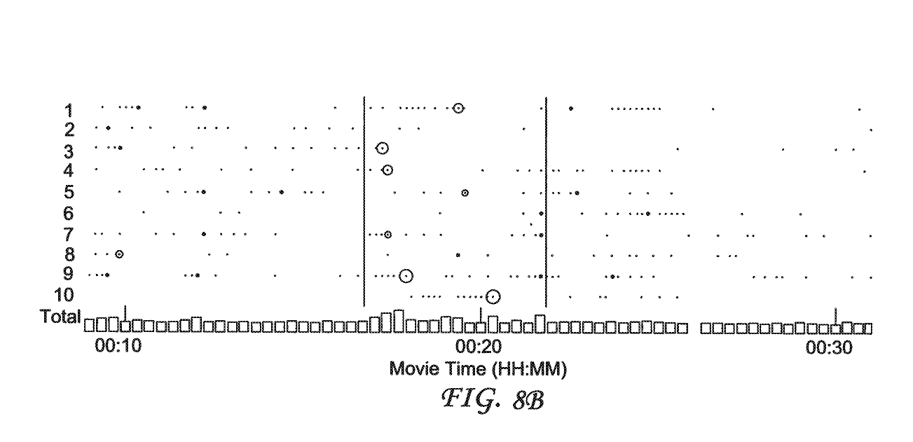

FIGS. 8A and 8B shows user responses as point intensities for two particularly relevant scenes from two movies, identified as Movie A and Movie B. As seen in both FIGS. 8A and 8 B, the SCR events appear generally sparse and vary considerably in their intensities. Furthermore, due to the physiological differences among the different users, the SCR events may not temporally align and could consist of spurious events not relevant to the stimuli in the film being watched.

To mitigate this inherent sparsity in the user response matrix S, the system 10 extracts the coarse-scale user response information by aggregating the information into a reduced number of time-aggregated bins. For each time bin, the system 10 records the sum of SCR coefficient energies for that time period. For the experiments described above, the system 10 combined the user SCR events over the course of the entire stimulus into five equal-sized bins, denoting the aggregated [N×5] user response matrix as S_{A}.

Combining the user response matrix S_{A} with the user demographic information yields a complete response matrix, S_{C}=[S_{A} C]. The matrix C comprises an [N×2] matrix constructed from the element C_{i,1} the gender of the user uᵢ and the element C_{i,2} the age of the user uᵢ

To solve the problem of inferring explicit user feedback information (e.g., film ratings), the system 10 will classify the decomposed user responses, S_{C}, using bagged classification trees. Bagged classification trees enable the system 10 to learn an ensemble of simple tree classifiers over multiple subsamples of a held-out training set. Specifically, to classify a particular user's rating, the system 10 uses leave-one-out cross validation such that the EDA signals from remaining users remain as training data only. From this collection of training data, the system 10 chooses a random subsample of training users and learns a single classification tree with respect to that training subset ground truth. For example, the system 10 may learn that if the response energy in the first time bin lies below a learned value, then the user will rate the film poorly. During each iteration, the system 10 will learn weights with respect to the classification accuracy on the training set in addition to learning the classification tree. Ultimately, the system 10 uses the specified test user data on a weighted combination of all the learned trees to classify the underlying explicit feedback for that user. The system 10 performs this bagged classifier approach on both the processed EDA data (the matrix S_{C}) and the demographics-only information (the matrix C).

The foregoing describes a technique for assessing users' responses to content in accordance with electro-dermal activity signals.

1. A method for determining user responses to content, comprising the steps of:
collecting Electro-Dermal Activity (EDA) signals from a user via a collection system as the user consumes content;
extracting from the collected EDA signals, the amplitudes of the users' responses at particular times;
processing the extracted amplitudes with demographic information for the user and parameters of the collection system obtained during training to predict feedback of the user to the content.

2. The method according to claim 1 wherein the amplitudes of the users' responses are extracted using one of deconvolution, change-point detection, or adaptive decomposition.

3. The method according to claim 1 wherein processing the extracted amplitudes include aggregating the extracted signal amplitudes for pre-determined time segments.

4. The method according to claim 3 wherein the processing step includes the step of applying ensemble tree classification to the aggregated signals, the demographic information for the user and the parameters of the collection system obtained during training to predict the user feedback.

5. The method according to claim 1 wherein the parameters for the collection are obtained during training by the steps of:
collecting Electro-Dermal Activity (EDA) signals from a user via a collection system as the user consumes pre-selected content;
extracting from the collected EDA signals, the amplitudes of the users' responses at particular times; and
performing ensemble tree classification on the extracted EDA signal amplitudes to yield the parameters.

6. A system for determining user responses to content, comprising a processor for (1) collecting Electro-Dermal Activity (EDA) signals from a user as the user consumes content; (2) extracting from the collected EDA signals which are amplitudes of users' responses at particular times; and (3) processing the extracted amplitudes with demographic information for the user and parameters obtained during training to predict feedback of the user to the content.

7. The system according to claim 6 wherein the processor extracts the amplitudes of the users' responses using one of deconvolution, change-point detection, or adaptive decomposition.

8. The system according to claim 7 wherein the processor processes the extracted amplitudes by aggregating the extracted signal amplitudes for pre-determined time segments.

9. The system according to claim 8 wherein the processor applies ensemble tree classification to the aggregated signals, the demographic information for the user and the parameters of the collection system obtained during training to predict the user feedback.

10. The system according to claim 6 wherein the processor determines parameters during training by executing computer instructions for:
collecting Electro-Dermal Activity (EDA) signals from a user via a collection system as the user consumes pre-selected content;
extracting from the collected EDA signals, the amplitudes of the users' responses at particular times; and
performing ensemble tree classification on the extracted EDA signal amplitudes to yield the parameters.

11. The system according to claim 10 wherein the processor constructs the signal component dictionary by parameterizing dictionary basis functions as follows:
$${d}_{{λ}_{1} , {λ}_{2} , {t}_{0}} ( t ) = \{ \begin{matrix}{λ}_{1}^{- {λ}_{2} ( t - {t}_{0} )} & t \geq {t}_{0} \\ 0 & t < {t}_{0}\end{matrix} \qquad$$
( such that λ₁ relates to a geometric decay of an impulse, λ₂ constitutes a log-linear decay slope, and t₀ corresponds to a response start, and
constructing the signal dictionary occurs using all signals for a parameter space,
λ₁ε{1.1,1.25,1.5,1.75,2,2.5,e},
λ₂ε{0.3,0.5, . . . ,3.7,3.9}.
